(19) United States
(12) Patent Application Publication
(10) Pub. No.: US 2023/0301525 A1
(43) Pub. Date: Sep. 28, 2023

(54) A System And Method For Thermometric Normalisation Of Blood Pressure Measurements

(71) Applicant: USCOM Limited, Sydney, New South Wales (AU)

(72) Inventor: Robert Allan Phillips, Coffs Harbour, New South Wales (AU)

(21) Appl. No.: 18/040,749

(22) PCT Filed: Aug. 5, 2021
(86) PCT No.: PCT/AU2021/050853

§ 371 Date: Feb. 6, 2023

(30) Foreign Application Priority Data
Aug. 6, 2020 (AU) 2020902770

(51) Int. Cl. A61B 5/021; A61B 5/01; A61B 5/00; A61B 5/02; A61B 5/029

(52) U.S. Cl. CPC A61B 5/02108; A61B 5/01; A61B 5/7271; A61B 5/02028; A61B 5/029; A61B 2560/0252

(57) ABSTRACT

A method of determining an accurate measure of blood pressure, the method including the steps of: initially measuring a patient's blood pressure measurement; determining a temperature measurement of the surrounds where the pressure measurement was obtained; and modifying or normalising the blood pressure measurement by a correction factor as determined by the temperature measurement.

### FIELD OF THE INVENTION

The present invention relates to a system and method for thermometric normalisation of blood pressure measurements and includes a method and system for improving the application of transcutaneous and intra-arterial BP monitoring.

### BACKGROUND OF THE INVENTION

Any discussion of the background art throughout the specification should in no way be considered as an admission that such art is widely known or forms part of common general knowledge in the field.

Arterial blood pressure (BP) is a fundamental measure of cardiovascular performance. Blood pressure measured in the brachial artery is the most common clinical observation and is a simple and accessible measure of health. Blood pressure changes in disease, with high and low BP significantly predicting death, stroke and heart attack and ultimately death. Hypertension, high BP, is the most common preventable cause of cardiovascular disease. While the exact values of upper normal BP vary, a brachial BP of 140/80 is considered the upper limit for normal, with some recommendations suggesting that the systolic BP or mean arterial BP (MAP) are the most predictive of cardiovascular risks.

Blood pressure measurement is currently commonly performed manually by auscultation or oscillometry on the brachial artery, a small artery immediately above the elbow. However, both methods have proven to poorly agree with invasive manometric measures of arterial BP, and neither agrees with central BP, the BP at the heart which is the gold standard measure that best predicts CV complications.

While hypertension occurs in approximately 70% of people over the age of 35 years, management of BP is effective in <50% of cases, suggesting an error in the measurement technology, the measurement protocol, or a misunderstanding of the pathophysiology leading to poor targeting of therapy, or all of these factors.

The function of the heart and vessels are co-ordinated to optimise delivery of oxygen, bound to red blood cells and transported in the blood to the cells of the body. The blood flows at a volume and pressure controlled by the heart and vessels respectively, while the BP is the product of both cardiac and vascular function, i.e. MAP=(SV×HR)×SVR.

Further, optimal management of BP is dependent on optimising cardiac function (SV and HR) and vascular function measured as Systemic Vascular Resistance (SVR). To add further complexity, cardiac and vascular function are interdependent and controlled by the autonomic nervous system (ANS) mediated by baro-receptors (pressure), therm oreceptors (temperature) and chemoreceptors (oxygen and CO2). Changes in BP, temperature (T) and oxygen delivery result in ANS modulated regulation.

The vascular network consists of ramifying vessels from the large arteries leading out of the heart, to small arteries, arterioles, capillaries (exchange vessels), venules, small veins and large veins that form a linear conduit transporting the blood around the body. The vessels are composed of a thin intimal layer, a thicker mid smooth muscle layer and the externa. The smooth muscles of the vessels are the functional part of the circulation constricting or dilating under control of the ANS. The SVR is a major and dynamic component of BP responding beat to beat to small changes in the vascular tone, with vaso-constriction increasing the SVR and vaso-dilation, relaxation, decreasing the SVR.

The skin is an extensive and dynamic organ weighing as much as 2 kg with a surface area in the order of 1.8 m². One of its principal functions is heat regulation with anatomy characterised by a dense system of capillary loops that empty into a capacious sub-capillary venous plexus. Humans are unique in that their response to heat stress almost entirely involves active vasodilation and sweating. While normal skin temperature is ˜32°, local cooling of the skin can reduce blood flow to zero, while skin temperature of >40° can result in a 5 to 10-fold increase in blood flow, representing direct effects of heat on vascular smooth muscle.

Rowell reported that baseline total skin blood flow is ˜200 to 500 ml/min. (Rowell, L B. Human Circulation Regulation during Physical Stress. New York (NY): Oxford University Press; 1986. Thermal stress; p. 174-212, Rowell L B, Brengelmann G L, Murray J A. Cardiovascular responses to sustained high skin temperature in resting man. J Appl Physiol 1969; 27:673-80. [PubMed: 5360442]). Maximally vasodilated skin during exercise to the limits of thermal tolerance receives flow up to 7-8 L/min, a ˜30-fold increase. Crandall et al found that during heat stress carotid-cardiac baroreflex did not vary significantly yet carotid-vascular baroreflex was reduced by ˜35% suggesting the heart remained unregulated, while the vessels were actively dilated in response to heat, with this vasodilation resulting in the skin flow shifting from 1-2% to >50% of totals CO during maximal exercise.

More directly Rowell demonstrated that during upright exercise an increase in skin temperature from 32° to 38°, effectively dilating the cutaneous plexus, reduced the central blood volume, the SV, the SVR, and MAP (reduced from 95 mmHg to 85 mmHg, −10 mmHg and −11%). Conversely a decrease in skin temperature from 38° to 27° produced an increase in central blood volume, SV, SVR and MAP (from 85 to 100 mmHg, +15 MmmHg or +18%). Jones et al. reported that an inaccuracy of 5 mm Hg in the measurement of BP was estimated to result in the misclassification of BP status of 48 million people each year in the United States alone, with 21 million underestimated BP, and 27 million overestimated BP. (Jones D W, Appel L J, Sheps S G, Roccella E J, Lenfant C. Measuring blood pressure accurately: new and persistent challenges. JAMA 2003; 289: 1027-30.).

### SUMMARY OF THE INVENTION

It is an object of the invention, in its preferred form to provide a system and method for more accurate monitoring of blood pressure measurements.

In accordance with an aspect of the present invention, there is provided a method of determining a more accurate measure of blood pressure, the method including the steps of: initially measuring a patient's blood pressure measurements; determining a temperature measurement that the pressure measurement was obtained; and modifying or normalising the blood pressure measurement by a correction factor determined by the temperature measurement.

In some embodiments, the correction factor is inversely proportional to temperature.

In accordance with an aspect of the present invention, there is provided a system for measuring a patient's blood pressure, the system including: initial blood pressure measurement system for determining an initial patient blood pressure value; a temperature sensor for sensing a temperature measure associated with the environment in which the initial blood pressure measurement was taken; and an adjustment calculation means adjusting the initial patient blood pressure value in accordance with the detected temperature measure to output a final blood pressure measure.

### BRIEF DESCRIPTION OF THE DRAWINGS

Embodiments of the invention will now be described, by way of example only, with reference to the accompanying drawings in which:

FIG. 1 illustrates an example environment for a monitoring system created in accordance with a first embodiment;

FIG. 2 illustrates the processing flow of the embodiment of FIG. 1;

FIG. 3 illustrates the Average monthly minimum and maximum temperatures in Boston, the nearest weather centre to Framingham, demonstrating significant diurnal and annual variance of ambient temperature.

FIG. 4 illustrates a Model of thermometrically normalised systolic blood pressure (TNBPs) demonstrates TNBPs values in mmHg/° C. increasing with increasing systolic BP and T° C. A normal TNBPs <7 mmHg/° C., being 140 mmHg at 20° C.;

FIG. 5 illustrates a Model of thermometrically normalised diastolic blood pressure (TNBPd) demonstrates TNBPd values in mmHg/° C. increasing with increasing systolic BP and T° C. A normal TNBPd <4 mmHg/° C., being 80 mmHg at 20° C.; and

FIG. 6 illustrates a Model of thermometrically normalised mean arterial blood pressure (TNBP MAP) demonstrates TNBP MAP values in mmHg/° C. increasing with increasing systolic BP and T° C. A normal TNBP MAP <5 mmHg/° C., being 100 mmHg at 20° C.

### DETAILED DESCRIPTION OF THE INVENTION

The embodiments take advantage of the observation that vascular and cardiovascular function changes significantly with ambient temperature (T), and that ambient T complicates the prediction of cardiovascular risk from BP measurements alone. Thus, the same subject measured at different temperatures (T) will have different vascular function and different measured BP's, confounding the prediction of CV risk. The embodiments provide a method of normalising BP measures to ambient temperature T to create a thermometrically normalised measure of cardiovascular performance which may improve cardiovascular risk prediction to that provided by BP measures alone.

The embodiments describe the background physiology, modelling and insights that led to the development of new algorithms to reflect normalisation of BP measures to ambient temperature T. The algorithms are used to generate thermometrically normalised BPs, BPd and BP MAP and the parameters are labelled TNBPs, TNBPd, TNBP MAP. Importantly these new parameters are simply acquired and can be added to current measurement technologies and are relevant to transcutaneous and intra-arterial BP monitoring. These parameters also function to improve the personalisation of cardiovascular risk assessment by normalising for an additional independent variable.

The preferred embodiments utilise the fact that changes in temperature and skin blood flow can result in changes of 10 to 20 mmHg in the clinical measurement of MAP and may be clinically significant. The consequences of this thermometric variation are that a subject may be classified as normotensive although hypertensive, if measured at a high ambient temperature, or classified as being hypertensive while being normotensive if measured in the cold. This misclassification has an attendant personal and community cost associated with protracted unnecessary or ineffective therapy and has insurance ramifications of being diagnosed with a serious high risk cardiovascular disease—hypertension. Misclassification of hypertension status has significant clinical, therapeutic, social and economic implications.

It is apparent that the skin is a large and dynamic organ that responds to thermal regulation by changing the vascular resistance and shifting significant total blood volume from core to periphery and has a potential to significantly change blood pressure (BP) in response to low or high ambient temperatures. The substantial redistribution of blood flow associated with relatively small ambient T changes of <10° can change brachial BP measurements from 10-20 mmHg, values resulting in significant misdiagnosis and inappropriate therapy in hypertensive care.

The ambient temperature T therefore represents a crude analogue of the inverse SVR, with an increased temperature resulting in a decreased SVR, and a decreased T associated with an increased SVR. While SVR can be measured precisely as SVR=BP/SV×HR, and can be quantitated using Doppler ultrasound, ambient T is easily and reliably measured as an analogue of SVR. However, in some instances this interchangeability of SVR and T may not be absolute, particularly if the ANS is impaired. Optimally the measurement of both SVR and ambient T would be preferred.

In the cold, the peripheral vessels constrict, and in the heat they dilate, and, during the normal process of thermoregulation, the SVR changes, and thereby the measured BP. However, this change in measured BP may not reflect a change in cardiovascular risk, just a normal physiologic redistribution of blood volume associated with T variation.

### Anatomy and Physiology

The reserve or capacity of the cutaneous venous plexus is dependent on compliance of the veins (dV/dP), the reverse of resistance (dP/dV), in the regions of the veins, with different regions having different morphologic characteristics. These features will show general and regional individual normal variability and differ with age related changes and differing regional distribution of cardiovascular changes and disease.

While the studies provide information on the response of the ANS to heat in normal subjects, BP is most useful in monitoring the abnormal cardiovascular system. Abnormalities such as CAD, cardiomyopathy and hypertrophy as found in hypertension, all impair the ANS regulations, and so the heat response would be expected to be increased where the baroreceptor set point cannot be maintained. In such cases it would be expected that at different temperatures and with heat impaired baroreceptor control the BP difference would be increased making a single or serial BP measures of diminished value.

An additional complication with conventional oscillometry is the method itself. A simple cuff is fixed around the upper arm and inflated with air through a hollow tube connected to a pump and pressure sensor. The systolic and diastolic pressures are determined from the relative occlusion of the brachial artery. The pressure in the cuff is measured as an analogue of brachial artery pressure, which is extrapolated as an analogue of central aortic pressure. However, as the cuff encircles the upper arm and is inflated, it compresses the cutaneous venous plexus beneath the cuff forcing a blood re-distribution into the central circulation or the adjacent uncompressed venous plexus. The degree and region of distribution will vary between individuals, and so is another source of inter-subject variability of BP during oscillometry.

Thus, without wishing to be bound by theory, is it hypothesised that changes in ambient T increase vascular plexus pooling, decrease vascular tone and SVR, and therefore decrease BP, if SV is preserved or increased. While the SV/CO may be downregulated by autonomic regulation, it is unlikely to be downregulated to the same degree as the SVR where skin flow is increased 25-fold. This thermometric response may create erroneous classification or normality or abnormality of BP resulting in unnecessary treatment of normal physiology, or non-treatment of abnormal BP. The same errors may be duplicated when monitoring the effectiveness or ineffectiveness of therapies.

So if the BPs is 140 mmHg at 20° C., and as BP=(SV×HR)×SVR, and BP is normally at a baroreceptor “set point”, then as T increases to say 30°, then the peripheral vessels will dilate, SVR will decrease and the SV will be increased due to the reduced resistance and under the autonomic control the BP preserved. Conversely if the T is reduced, to say 10°, then the peripheral vessels will constrict and the SVR increase, and the ANS acts to reduce the SV and CO. So, in normal patients BP changes may be minimised by autonomic compensation. However, if the autonomic system is impaired or the baroreceptor “set points” changed due to a change in T, then the physiologic BP may be significantly change.

Thus, a BP of 140/80 at 30° C., in a state of relative vascular relaxation and vaso-dilation with a low SVR, is likely to be more significant than the same BP at 15° in a state of vasoconstriction where the SVR is elevated. Importantly, BP is used as a trend measure, with the election to treat based on the accuracy of the BP measure. Further, the effectiveness of therapies are determined by monitoring the changes in BP. Changed BP measures caused by changes in ambient T may result in both over therapy, under therapy or imprecise assessment of the effect of therapy. From the clinical perspective monitoring changes in BP, high, normal or low, will be improved by indexing the value to the ambient T. Importantly the same thermal re-distribution is relevant to transcutaneous and intra-arterial monitored BP.

### Example System

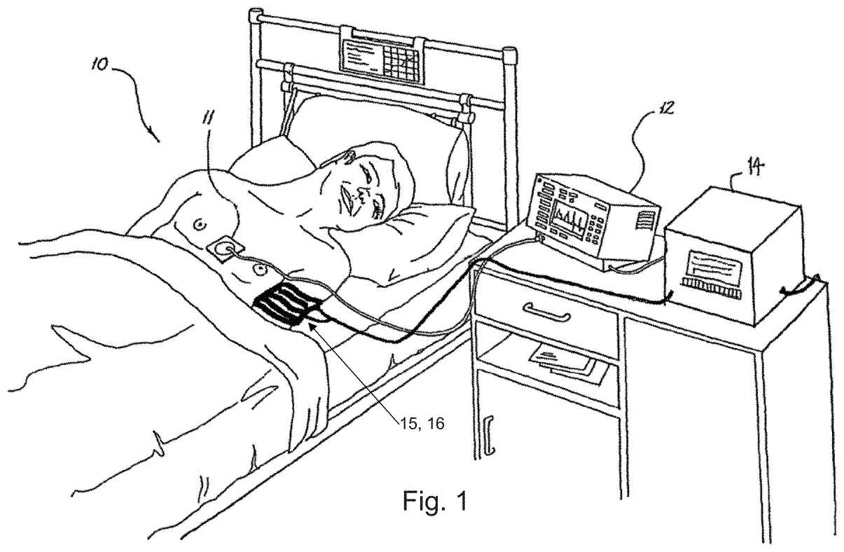

FIG. 1 illustrates one form of system environment 1 of an embodiment. In this environment, a patient 10 is in a hospital bed and has a pressure monitoring system 15, which is also modified to include a temperature sensor 16. These are interconnected to a control unit 14.

Additionally, the system includes an USCOM heart output monitor 11 and control unit 12, which monitor the heart's cardiac output using CW Doppler flow measurements. The principles of CW Doppler flow measurement are known. Patent Cooperation Treaty (PCT) publication number WO99/66835 to the present assignee, the contents of which are incorporated herein by cross-reference, describes in more detail an ultra-sonic transducer device suitable for measuring blood flow using the CW Doppler method.

In essence the temperature monitoring system is used to modulate the pressure values recorded as described hereinafter.

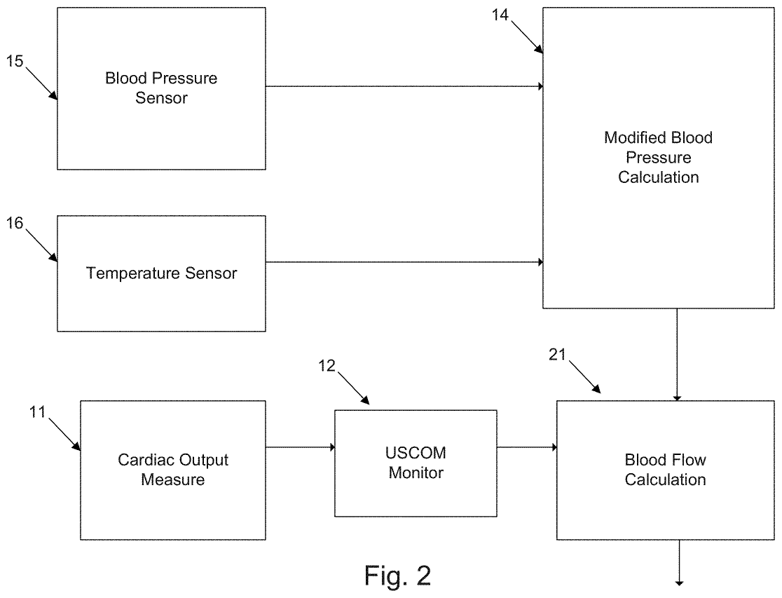

The processing arrangement can be as shown in FIG. 2, where blood pressure measurements 15 and temperature sensor measurements 16 are forwarded to modified blood pressure calculation unit 14 which calculates a modified blood pressure measurement which is output. The cardiac output 11 is also calculated by USCOM monitor 12 and forwarded to produce an overall measure of blood circulation.

### Ambient Temperature Variation and Hypertension

The clinical significance of ambient T variation can be demonstrated by considering the potential impact of temperature (T) variation in the Framingham data. Framingham is the site of the largest and most enduring reference database for hypertension compiled since 1958 and continuing as an on-going study on the incidence, evolution and outcomes of hypertension. Framingham, outside Boston, Massachusetts, USA, has a monthly mean maximum daily temperature ranging from 2° C. in January to 28° C. in July, with consistent annual diurnal range of 10° C. (FIG. 2). Therefore, if measuring BP at ambient temperature on subjects in Framingham, then both the date of measurement and the time of day of measurement will be relevant to vascular tone, SVR and BP values. While the presence of air-conditioning may mitigate such variance, the variation of ambient T's may have a short-term impact by causing transient vasoconstriction or vasodilation. The impact of this annual temperature variation may result in serial recalibration of the baroreceptor “set points” controlling the ANS such that the baseline SVR is different throughout the year. Both short and long-term influences effect the accuracy of BP measures, the classification of hypertension, and potentially confounds the reliable prediction of cardiovascular risk and optimal choice of therapy. Thermometric normalisation of BP measurements to ambient T controls for a significant environmental variable and may improve the effectiveness of BP monitoring, diagnosis and therapy. It may also improve the interpretation of the evidence and conclusions from the Framingham data.

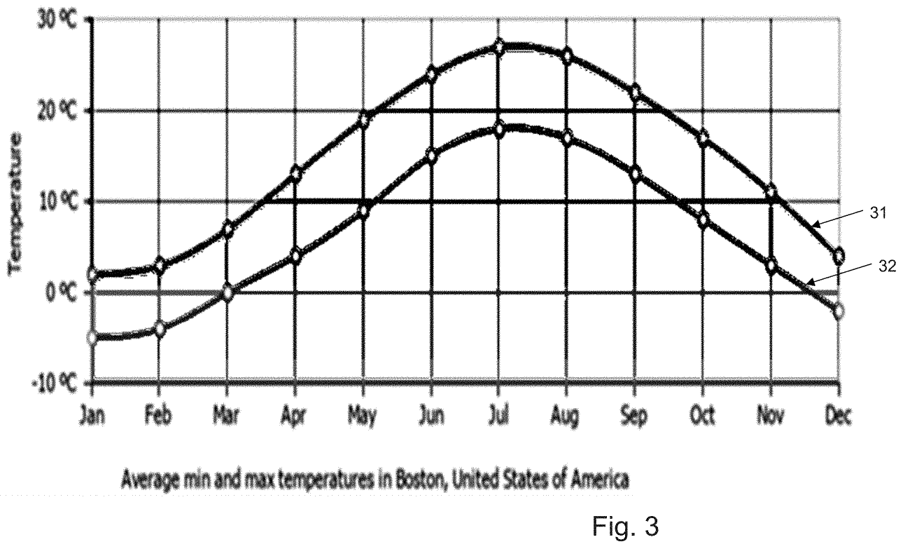

FIG. 3 illustrates Average monthly minimum 32 and maximum 31 temperatures in Boston, the nearest weather centre to Framingham, demonstrating significant diurnal and annual variance of ambient temperature.

The application of personally targeted precision medicine may also be improved using thermometric normalisation of BP measures. Variability of any set of BP measurement represents the sensitivity of the method, the variability of operators and their techniques and protocols, and the reliability of the technologies used, as well as the variability of the physiology—the parameter that is sought to be measured. By controlling for non-physiologic variables, such as temperature T, the sensitivity for detecting personal physiologic change should be increased.

### Example Normalisation Algorithms

Dividing the BPs, BPd and MAP by the ambient T in ° C. provides a normalised value that demonstrates an increasing risk at decreasing temperature. Thus, an upper limit for normal BPs of 140 mmHg has a different clinical significance at 30°, where the peripheral venous plexuses are dilated with a low SVR, then at 15° where they are constricted and SVR is elevated. While the method is precise, predicting the clinical consequence assumes fixed ANS function and response. The physiological variables are many and independently variable making simple modelling and predictions of their effect difficult. Most importantly is that the new measures are used to more sensitively detect the changes within each individual.

An example measurement of Ambient thermometric normalised peak systolic BP (TNBPs), can be as follows:

BPsys/T=Peak systolic arterial pressure divided by the ambient temperature in ° C. Below are thermometrically normalised systolic BP (TNBPs) of 140 mmHg at 15°, 20° and 30°.

TNBPs (15°)=140/15=9.3, or 15/140=0.11

TNBPs (20°)=140/20=7.0, or 20/140=0.14

TNBPs (30°)=140/30=4.7, or 30/140=0.21

TNBPs <6 is normal.

Ambient thermometric normalised peak diastolic BP (TNBPd).

BPd/T=Peak diastolic arterial pressure divided by the ambient temperature in ° C.

Below are thermometrically normalised diastolic BP (TNBPd) of 80 mmHg at 15°, 20° and 30°.

TNBPd (15°)=80/15=4.7, or 15/80=0.19

TNBPd (20°)=80/20=4.0, or 20/80=0.25

TNBPd (30°)=80/30=2.7, or 30/80=0.375

TNBPd<4 is normal.

Ambient thermometric normalised MAP (TNBP MAP).

MAP/T=Mean arterial pressure divided by the ambient temperature in ° C.

Below are thermometrically normalised MAP (TNMAP) of 100 mmHg at 15°, 20° and 30°.

TNMAP (15°)=100/15=6.7, or 15/100=0.15

TNMAP (20°)=100/20=5.0, or 20/100=0.2

TNMAP (30°)=100/30=3.4, or 30/100=0.3

TNMAP <5 is normal.

Ambient temperature normalised pulse pressure (BPs-BPd)

PP/T=pulse pressure divided by ambient temperature in ° C.

There may be considerable variability between individuals and their baroreceptor set points, meaning that at any temperature the normal BP will vary between individuals. In additional the ANS function of each person will vary between normal individuals, and across subjects with various types and degrees of cardiovascular diseases. So, any method which generates measures controlled for any variable, in this case ambient temperature, will provide for more sensitive detection of real physiologic change. This is essential for precision, as opposed to measurement of gross changes which may be related to variation in devices, measurement techniques and protocols, or real physiologic change.

As cardiac function can also vary with fluid volume and adrenergic stimulation, so normalisation to a normal SVV on Doppler ultrasound and following 10 minutes rest may also assist controlling for physiologic variables and make serial BP measures more precise and meaningful clinically.

### Graphical Representation of Thermometrically Normalised Blood Pressure Values

The plotting of BPs, BPd and BP MAP against ambient temperature and TNBP may provide improved understanding of the relationship between BP and temperature and provide an improved parameter for determining cardiovascular risk. These plots can also be used to define normal and abnormal values and monitoring changes associated with therapy.

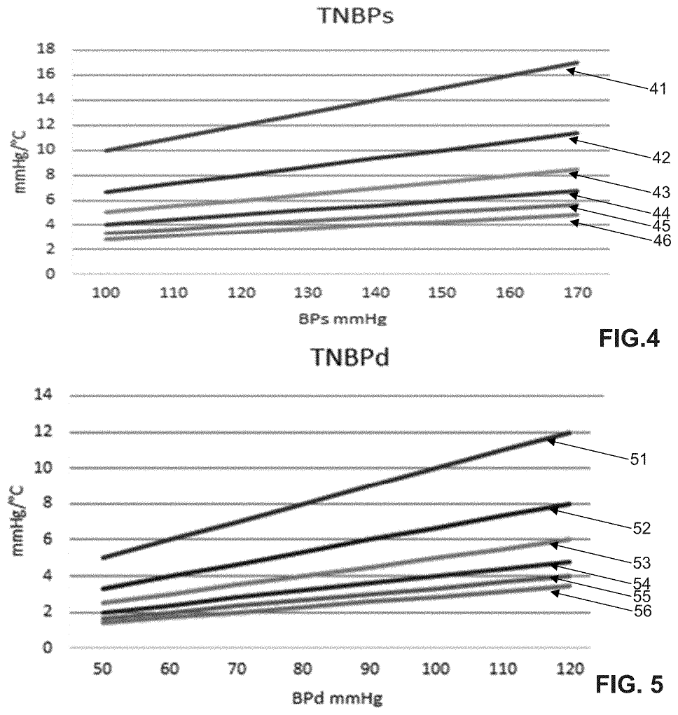

FIG. 4 illustrates a Model of thermometrically normalised systolic blood pressure (TNBPs) demonstrates TNBPs values in mmHg/° C. increasing with increasing systolic BP and T° C. A normal TNBPs <7 mmHg/° C., being 140 mmHg at 20° C. Temperature plots are shown from 10° C. to 35° C., in 5° C. increments 41-46.

FIG. 5 illustrates a Model of thermometrically normalised diastolic blood pressure (TNBPd) demonstrates TNBPd values in mmHg/° C. increasing with increasing systolic BP and T° C. A normal TNBPd <4 mmHg/° C., being 80 mmHg at 20° C. Temperature plots are shown from 10° C. to 35° C., in 5° C. increments 51-56.

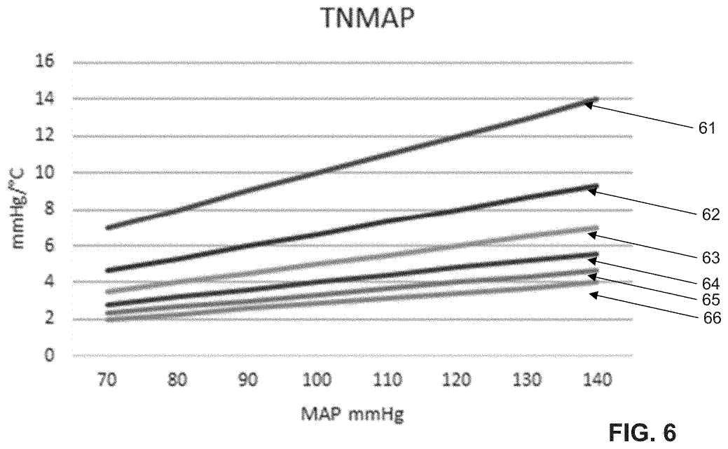

FIG. 6 illustrates a Model of thermometrically normalised mean arterial blood pressure (TNBP MAP) demonstrates TNBP MAP values in mmHg/° C. increasing with increasing systolic BP and T° C. A normal TNBP MAP <5 mmHg/° C., being 100 mmHg at 20° C. Temperature plots are shown from 10° C. to 35° C., in 5° C. increments 61-66.

Normal values:

TNBPs <7 mmHg/° C. (MAP 140 mmHg at 20° C.)

TNBPd <4 mmHg/° C. (MAP 80 mmHg at 20° C.)

TNBP MAP <5 mmHg/° C. (MAP 100 mmHg at 20° C.)

### Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method of determining an accurate measure of blood pressure, the method including the steps of:
initially measuring a patient's blood pressure measurement;
determining a temperature measurement of the surrounds where the pressure measurement was obtained; and
modifying or normalising the blood pressure measurement by a correction factor as determined by the temperature measurement.

2. A method as claimed in claim 1 wherein the correction factor is inversely proportional to temperature.

3. A method as claimed in claim 1 wherein said temperature measurement is an ambient temperature measurement.

4. A method as claimed in claim 1 wherein said temperature measurement is a measurement of the patient's body temperature.

5. A method as claimed in claim 1 further comprising measuring at least one of the stoke volume, heart rate or the systemic vascular resistance (SVR) of the patient and modifying the blood pressure measurement by a further correction factor determined by said measurements.

6. A system for measuring a patient's blood pressure, the system including:
initial blood pressure measurement system for determining an initial patient blood pressure value;
a temperature sensor for sensing a temperature measure associated with the environment in which the initial blood pressure measurement was taken; and
an adjustment calculation means adjusting the initial patient blood pressure value in accordance with the detected temperature measure to output a final blood pressure measure.

7. A system as claimed in claim 6 further including:
blood measurement system adapted to measure at least one of stroke volume, heart rate or systemic vascular resistance, and
said adjustment calculation means adjusting the final blood pressure measure by a factor determined by the blood measurement system.
